(19) United States
(12) Patent Application Publication
(10) Pub. No.: US 2021/0186751 A1
(43) Pub. Date: Jun. 24, 2021

(54) SYSTEM FOR HYPOTHERMIA TREATMENT OF A PATIENT

(71) Applicant: Quickcool AB, Lund (SE)

(72) Inventors: Mats ALLERS, Lund (SE); Anders LUNDERQUIST, Lund (SE)

(73) Assignee: Quickcool AB, Lund (SE)

(21) Appl. No.: 16/755,165

(22) PCT Filed: Jan. 18, 2019
(86) PCT No.: PCT/EP2019/051249

§ 371 Date: Apr. 10, 2020

(30) Foreign Application Priority Data
Jan. 23, 2018 (SE) 1850069-4

(51) Int. Cl. A61F 7/12; A61B 17/132; A61H 39/04; A61H 7/00; A61M 19/00; A61M 25/02

(52) U.S. Cl. CPC A61F 7/12; A61B 17/1325; A61H 39/04; A61B 2017/12018; A61M 19/00; A61M 25/02; A61H 7/006

(57) ABSTRACT

A constriction device (100) adapted to be placed on the head of a patient for regulating the temperature of the brain. The constriction device comprises at least two protruding pressure elements (101a′, 101b′, 101c′) configured to create bilateral pressure on at least one from a list consisting of: the facial veins (10), the superficial temporal veins (20), the posterior auricular veins (30), and the occipital veins (40), such that the blood flow in the constricted veins is restricted.

### FIELD OF THE INVENTION

The present invention relates to a device for use in hypothermia treatment of a patient.

### BACKGROUND

Blood supply to the brain is normally divided into anterior and posterior segments, relating to the different arteries that supply the brain. The two main pairs of arteries are the carotid arteries (arteria carotis interna, arteria carotis externa), supplying the anterior of the brain, and the vertebral arteries (arteria vertebralis), supplying the brainstem and the posterior brain. (see for example Moore et al. “clinically oriented Anatomy”, 1999). The anterior and posterior cerebral circulations are interconnected via bilateral posterior communicating arteries. They are part of the Circle of Willis (circulus arteriosus Willisii), which provides backup circulation to the brain. In case one of the supply arteries is occluded, the Circle of Willis provides interconnections between the anterior and the posterior cerebral circulation.

The venous drainage of the cerebrum can be separated into two subdivisions: superficial and deep. The superficial venous system is composed of dura mater and is located on the surface of the cerebrum. The most prominent portion of the superficial venous system is the superior sagittal sinus (sinus sagittalis superior) which flows in the sagittal plane (centrally between the two brain halves) to the confluence of sinuses (confluens sinuum), where the superficial sagittal sinus joins with the inferior sagital sinus (sinus sagittalis inferior) that primarily drains the deep venous system. From here, the superior sagittal sinus and the inferior sagittal sinus bifurcate and travel laterally forming the internal jugular veins (vena jugularis interna).

The superficial venous system is connected to the extracranial venous system of the scalp via emissary veins (vena emisaria). The emissary veins drain blood from the scalp, through the skull, and into the superior sagittal sinus. Because the emissary veins are valveless, they are an important part in selective brain cooling through bidirectional flow of cooler blood from the scalp and warmer blood from the inside of the scull (for example shown by Baker 1982 and Caputa 1980). As shown by Cabanac and Brinnel in 1985, during hyperthermia cooler blood flow rapidly from scalp to brain in the emissary veins, cooling the brain, and during hypothermia blood flow slowly from the brain to the scalp causing the brain to retain more of the heat absorbed from the arterial blood supply.

The blood vessels in the scalp runs in the dense subcutaneous layer between the skin and the epicranial aponeurosis (aponeurosis epicranialis). The arterial supply of the scalp comes from the external cartotid arteries through the occipital, posterior auricular, and superficial temporal arteries (arteria occipitalis, arteria auricularis posterior, arteria temporalis superficialis) and from the internal cartotid arteries through the supratrochler and supraorbital arteries (arteria supratrochlearis, arteria supraorbitalis).

The facial vein (vena facialis) drains venous blood originating at the supraorbital vein (vena supraorbitalis) and the supratrochlear vein (vena supratrochlearis), which begin in the forehead and descend to unite at the medial angle of the eye to form the angular vein (vena angularis) that becomes the facial vein at the inferior margin of the orbit.

The superficial temporal vein (vena temporalis superficialis) drains the scalp anterior to the auricles and descends into the retromandibular vein (vena retromandibularis). The occipital vein (vena occipitalis) drains the occipital region of the scalp and empties into the internal jugular vein and the posterior auricular vein (vena auricularis posterior) drains the scalp posterior to the auricles and empties into the external jugular vein (vena jugularis externa). Further, the posterior auricular vein connects to the mastoid emissary vein (vena emissaria mastoidea) from the sigmoid sinus (sinus sigmoideus).

Temperature control of the brain can have both prophylactic and therapeutic effects in various conditions. E.g. in the case of a circulatory arrest in excess of 5-15 minutes, the brain can suffer permanent damage. However, if the temperature of the brain is lowered before, during or after the arrest, the risk of brain damage can be substantially reduced.

There are various ways that the brain can be cooled presented in the art. The various ways include full body cooling, retro-perfusion and retro-infusion cooling (such as described in WO 98/23217, and the cooling of cerebral arteries by the cooling of the sinus cavernous, such as further described in WO2005/087156 (to Lunderqvist and Allers).

However, as shown by Cabanac and Brinnel in 1985, induced hypothermia creates a response by the body to counter the effects of the cooling. This response includes altering the direction of the flow of venous blood in the emissary veins, such that the blood will start to flow from the superficial venous system to the scalp, which reduces the effect of for example cooling of the sinus cavernous.

### SUMMARY

A constriction device adapted to be placed on the head of a patient for regulating the temperature of the brain is provided. The constriction device comprises at least two protruding pressure elements configured to create bilateral pressure on at least one of: the facial veins, the superficial temporal veins, the posterior auricular veins, and the occipital veins. The constriction device is configured to constrict the veins such that the blood flow in the constricted veins is restricted.

The gentle constriction of the external veins causes cooler blood to be transported from the scalp and face to the venous drainage of the cerebrum and thus cooling the brain before flowing into the internal jugular vein. Further, as the brain is cooled before the body, the hypothalamic set point for temperature is lowered, which reduces the risk that the patient will start to shiver as a response to the induced hypothermia. The lowering of the hypothalamic set point thus reduces the need to counter the shivering using sedative drugs.

According to one embodiment, the constriction device is configured to create bilateral pressure on at least two of: the facial veins, the superficial temporal veins, the posterior auricular veins, and the occipital veins. The constriction device is configured to constrict the veins such that the blood flow in the constricted veins is restricted.

Creating bilateral pressure on more than one pair of veins may be advantageous as it allows for improved control over the cooling of the brain.

According to another embodiment, the constriction device is configured to create bilateral pressure on at least three of: the facial veins, the superficial temporal veins, the posterior auricular veins, and the occipital veins. The constriction device is configured to constrict the veins such that the blood flow in the constricted veins is restricted.

According to another embodiment, the constriction device is configured to create bilateral pressure on the facial veins, the superficial temporal veins, the posterior auricular veins, and the occipital veins, such that the blood flow in the constricted veins is restricted.

According to another embodiment, the constriction device comprises at least four protruding pressure elements spaced apart, such that areas where pressure is created may be alternated by areas in which no pressure is created.

According to another embodiment, the constriction device comprises at least six protruding pressure elements spaced apart.

According to one embodiment, the constriction device is configured to least partially encircle the head of the patient. The partial encircling of the head makes it possible for the constriction device to withhold the counterforce created by the protruding pressure elements.

According to one embodiment, the constriction device comprises a support structure to which the protruding pressure elements are connected. The support structure may at least partially encircle the head of the patient.

The support structure may be advantageous as it allows for keeping the protruding pressure elements in place at an appropriate pressure while at the same time being ergonomical for the patient.

According to one embodiment the support structure is elastic and/or comprises an elastic member. The elastic member may be a mechanical elastic member, such as a spring, or it may be a pneumatic or hydraulic elastic member. The protruding pressure elements in any of the embodiments may be configured to create the bilateral pressure by means of elastic force exerted by at least one of the elastic member and the elastic support structure.

According to one embodiment, the elastic support structure is configured to wholly or partially encircle the head of the patient.

According to one embodiment, the constriction device further comprises an adjustment device for adjusting the length of the support structure.

According to one embodiment, the at least two of the protruding pressure elements are movably mounted to the support structure, such that the position of the at least two protruding pressure elements can be adjusted in relation to each other, for positioning the protruding pressure elements in relation to the veins to be constricted.

According to one embodiment, the protruding pressure elements and/or the elastic support structure and/or the elastic member are configured to create a pressure on the veins exceeding 2 mm Hg.

According to one embodiment, the protruding pressure elements and/or the elastic support structure and/or the elastic member are configured to create a pressure on the veins exceeding 4 mm Hg.

According to one embodiment, the protruding pressure elements and/or the elastic support structure and/or the elastic member are configured to create a pressure on the veins exceeding 6 mm Hg.

According to one embodiment, the constriction device further comprises at least one fixation element adapted to fixate tubing to the constriction device. The one or more fixation element may be pivotable around a fixation point, which enables the tubing to be positioned in a suitable way for the patient and medical staff.

According to one embodiment, the at least one fixation element is detachably fixated to a fixation point, such that the fixation element can be easily removed and/or exchanged. The detachable fixation may for example comprise a pushbutton.

### BRIEF DESCRIPTIONS OF THE DRAWINGS

The invention will by way of example be described in more detail with reference to the appended drawings, which shows embodiments of the invention.

FIG. 1 shows a plain view of the venous system on the left side of the head of a patient.

FIG. 2 shows an elevated perspective view of the venous drainage of the cerebrum of a patient.

FIG. 3 shows a plain view of the right side of the head of a patient, when a constriction device according to a first embodiment has been positioned.

FIG. 4 shows a plain view of the right side of the head of a patient, when a constriction device according to a second embodiment has been positioned.

FIG. 5 shows a detailed schematic side view of a protruding pressure element according to a first embodiment.

FIG. 6 shows a detailed schematic side view of a protruding pressure element according to a second embodiment.

FIG. 7 shows a detailed schematic side view of a protruding pressure element according to a third embodiment.

FIG. 8 shows a plain view of the right side of the head of a patient, when a constriction device according to a third embodiment has been positioned.

FIG. 9 shows a more detailed view of the constriction device according to the third embodiment.

### DETAILED DESCRIPTION

The present invention is based on the discovery that venous blood flow of the scalp can be redirected towards the emissary veins by the gentle constriction of the external veins that drains the scalp and face, in particular the facial vein, the superficial temporal vein, the occipital vein and the posterior auricular vein. The gentle constriction of the external veins causes cooler blood to be transported from the scalp and face to the venous drainage of the cerebrum and thus cooling the brain before flowing into the internal jugular vein.

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided for thoroughness and completeness, and fully convey the scope of the invention to the skilled person. In the figures illustrating the venous system of the human head, smaller branches and veins which are of lesser importance to the invention have been omitted.

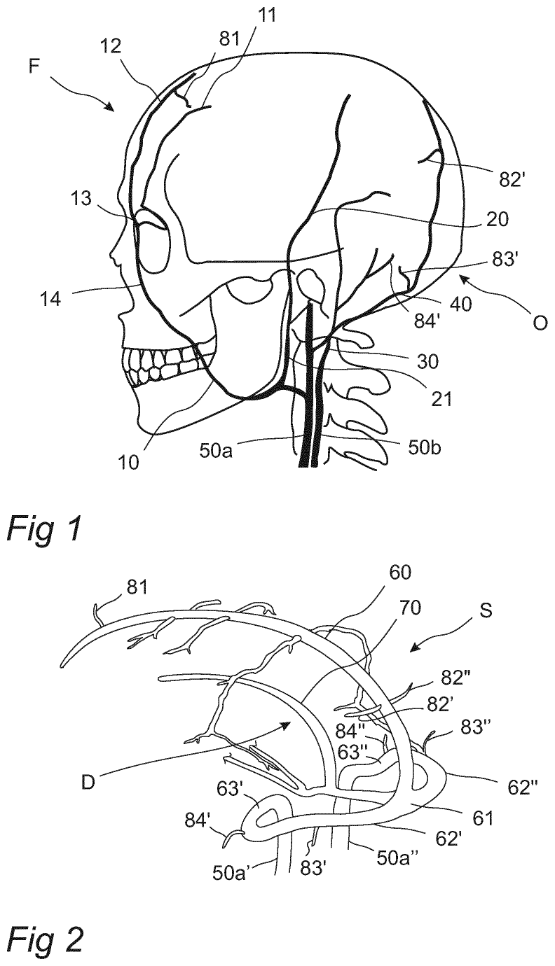

FIG. 1 shows the veins of the scalp that runs in the dense subcutaneous layer between the skin and the epicranial aponeurosis. The facial vein 10 drains venous blood originating at the supraorbital vein 11 and the supratrochlear vein 12, which begin in the forehead F and descend to unite at the medial angle of the eye 13 to form the angular vein 14 that drains into the facial vein 10.

The superficial temporal vein 20 drains the scalp anterior to the auricles and descends into the retromandibular vein 21, which unites with the facial vein 10. The posterior auricular vein 30 drains the scalp posterior to the auricles and empties into the external jugular vein 50b. The occipital vein 40 drains the occipital region O of the scalp and empties into the internal jugular vein 50a′.

FIG. 2 shows the venous drainage of the cerebrum, which can be separated into two subdivisions, the superficial drainage S and the deep drainage D. The superficial venous system S is composed of dura mater and is located on the surface of the cerebrum. The most prominent portion of the superficial venous system S is the superior sagittal sinus 60 which flows in the sagittal plane, centrally between the two halves of the cerebrum, to the confluence of sinuses 61. At the confluence of sinuses 61 the superior sagittal sinus 60 confludes with the inferior sagital sinus 70, that primarily drains the deep venous system D. From the confluence of sinuses 61, the superior sagittal sinus 60 and the inferior sagittal sinus 70 bifurcate and travel laterally forming the transverse sinuses 62′, 62″ which then becomes the sigmoid sinuses 63′, 63″ which then empties into the internal jugular veins 50a′, 50a″.

The superficial venous system S is connected to the extracranial venous system of the scalp via emissary veins. The emissary veins drain blood from the scalp, through the skull, and into the superior sagittal sinus 60, the transverse sinuses 62′, 62″ and the sigmoid sinuses 63′, 63″. The size and number of emissary veins very between individuals. A frontal emissary vein 81 is present in children and some adults. The frontal emissary vein 81 passes through the foramen cecum of the skull and connects the frontal portion of the superior sagittal sinus 60 with the venous plexus of the forehead F which drains into the supratrochlear vein 12 and the superficial temporal vein 20. The bilateral parietal emissary veins 82′, 82″ passes through the parietal foramina in the parietal portion of the skull connecting the dorsal portion of the superior sagittal sinus 60 with the venous plexus of the parietal region which drains into the occipital vein 40. The bilateral mastoid emissary veins 83′, 83″ passes through the mastoid foramen and connects each transverse sigmoid sinus 62′,62″ with the occipital vein 40 or the posterior auricular vein 30. Additionally, a posterior condylar emissary vein 84′, 84″ may be present, passing through the condylar canal, connecting the sigmoid sinuses 63′, 63″ with the suboccipital plexus of veins.

During hyperthermia cooler blood flow rapidly from scalp to brain in the emissary veins, cooling the brain, and during hypothermia blood flow slowly from the brain to the scalp causing the brain to retain more of the heat absorbed from the arterial blood supply. By gently constricting the major veins that drains the scalp, the blood flow can be reversed, such that cooler blood will flow inwards and lower the temperature of the brain.

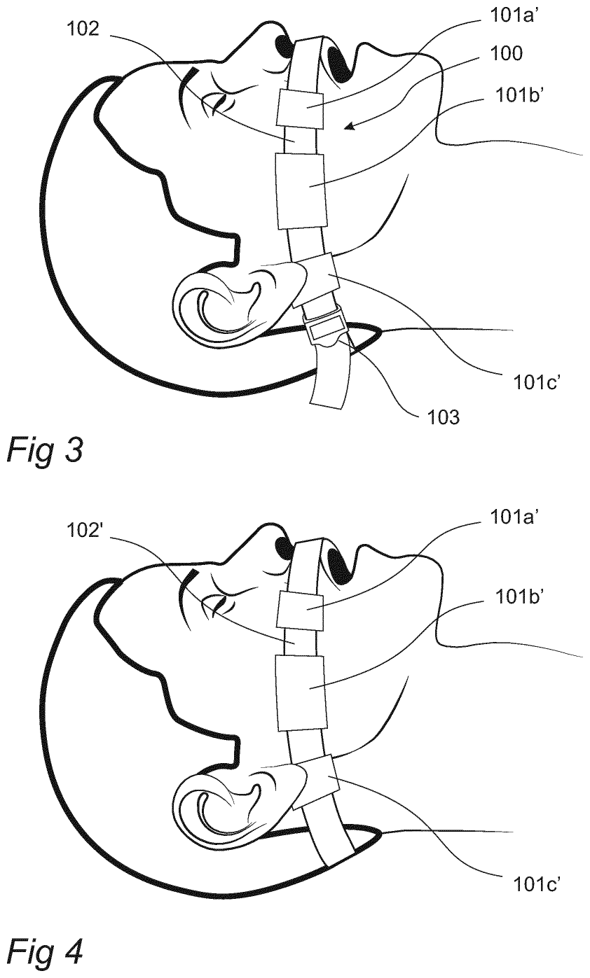

FIG. 3 shows a constriction device 100 adapted to be placed on and encircle the head of a patient for regulating the temperature of the brain by gently constricting veins and thereby altering the direction of the flow of blood from the scalp. The constriction device 100 of the embodiment shown in FIG. 3 comprises a total of six protruding pressure elements, three of which 101a′, 101b′, 101c′ are positioned on the right side of the patient's head, and three of which are positioned on the left side of the patient's head (hidden). The first protruding pressure elements 101a′ are configured to create bilateral pressure on the facial veins (14 in FIG. 1), the second protruding pressure elements 101b′ are configured to create bilateral pressure on the superficial temporal veins (20 in FIG. 1) and the third protruding pressure elements are configured to create bilateral pressure on the posterior auricular veins (30 in FIG. 1) and the occipital veins (40 in FIG. 1). The protruding pressure elements 101a′, 101b′, 101c′ are configured to create a pressure high enough such that the blood flow in the constricted veins is restricted. The venous pressure in the relevant veins is about 2 mmHg (≈267 Pa), and in the embodiment shown in FIG. 3, the protruding pressure elements 101a′, 101b′, 101c′ are configured to create a pressure on the veins exceeding 2 mmHg, thus constricting the vein and reducing the blood flow therethrough. The protruding pressure elements may however be configured to exert a pressure exceeding that in the veins, such as a pressure exceeding 4 mmHg, or a pressure exceeding 6 mmHg.

In the embodiment of the constriction device shown in FIG. 3, the protruding pressure elements 101a′, 101b′, 101c′ are spaced apart allowing air to flow between the protruding pressure elements 101a′, 101b′ 101c′. The constriction elements are spaced apart with a distance exceeding 1 mm, or a distance exceeding 3 mm, or a distance exceeding 7 mm.

In the embodiment of the constriction device shown in FIG. 3, the protruding pressure elements 101a′, 101b′, 101c′ are mounted on and connected to a support structure 102, which in the embodiment of FIG. 3 is a flexible flat strap made from a polymer material. The constriction device of FIG. 3 further comprises an adjustment device 103 for adjusting the length of the support structure 102. The adjustment device 103 of the support structure could be solely for the purpose of adjusting the length of the support structure to fit the individual patient, or for the additional purpose of assisting in the creation of the pressure exerted by the protruding pressure elements 101a′, 101b′, 101c′. The adjustment device 103 of FIG. 3 is in the form of a self-locking strap buckle 103.

In the embodiment shown in FIG. 3, the first protruding pressure element 101a′ has a length, in the direction of the length axis of the extension of the support structure, exceeding 10 mm. The second protruding pressure element 101b′ has a length, in the direction of the length axis of the extension of the support structure, exceeding 20 mm. The third protruding pressure element 101c′ has a length, in the direction of the length axis of the extension of the support structure, exceeding 10 mm.

In the embodiment of FIG. 3 four of the main external veins are constricted by means of three spaced apart protruding pressure elements 101a′, 101b′, 101c′. However, it is in alternative embodiments equally conceivable that the protruding pressure elements are made up of single longer protruding pressure elements (bilaterally) capable constricting all four veins. In such an embodiment, the single longer protruding pressure elements has a length, in the direction of the length axis of the extension of the support structure, exceeding 50 mm.

It is also conceivable that two of the three protruding pressure elements on each side are joined into a longer protruding pressure element capable of compressing two or three veins. In further alternative embodiments it is conceivable that not all four veins are compressed, i.e. the constriction device could comprise a single protruding pressure element capable of constricting for example only the facial vein.

FIG. 4 shows an alternative embodiment of a constriction device 100 adapted to be placed on and encircle the head of a patient for regulating the temperature of the brain. The embodiment of FIG. 4 is similar to the embodiment of FIG. 3, and the only difference is that the support structure 102′ is an elastic support structure 102′. The elastic support structure 102′ of FIG. 4 is a flat strap made from an elastomeric polymer material. Elasticity is to be understood as a materials ability to deform in an elastic way. Elastic deformation is when a material deforms under stress (e.g. external forces), but returns to its original shape when the stress is removed.

The elastic support structure 102′ could be solely for the purpose of allowing the length of the support structure 102′ to vary to fit the individual patient, or for the additional purpose of assisting in the creation of the pressure exerted by the protruding pressure elements 101a′, 101b′, 101c′. The elastic support structure 102′ in FIG. 4 is configured to exert a pressure on the protruding pressure elements 101a′, 101b′, 101c′, which in turn exerts a pressure on the veins exceeding 2 mmHg, thus constricting the veins and restricting the blood flow therethrough. In alternative embodiments, the elastic support structure may be configured to exert a pressure on the protruding pressure elements 101a′, 101b′, 101c′, which in turn exerts a pressure on the veins exceeding 4 mmHg or exceeding 6 mmHg.

In the embodiment of FIG. 4, the entire head-encircling elastic support structure 102′ is elastic. However, in alternative embodiments it is equally conceivable that only a portion of the support structure is elastic.

In the embodiments of FIGS. 3 and 4, the support structures 102, 102′ are entirely encircling the head of the patient. In alternative embodiments, it is however conceivable that the support structure is a support structure which does not wholly encircle the head. The support structure may instead have a C-shape, such as a diadem, which may be flexible, elastic or rigid.

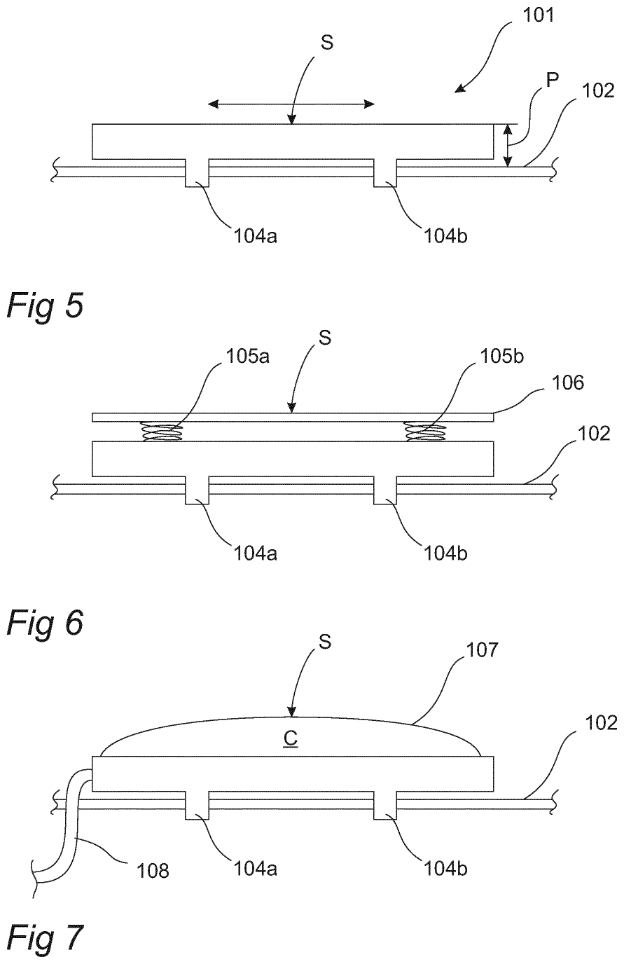

FIG. 5 shows a more detailed view of a protruding pressure element 101 according to a first embodiment. In the embodiment of FIG. 5, the protruding pressure element 101 is movably mounted to the support structure 102, such that the position of the protruding pressure element 101 can be adjusted. The adjustment of the position of the protruding pressure element 101 enables protruding pressure element 101 to be positioned at the correct location in relation to the vein which is to be compressed. The protruding pressure element 101 is movably mounted to the support structure by means of two mounting members 104a, 104b, snuggly encircling the support structure 102. In the embodiment of FIG. 5, the protruding pressure element protrudes a distance P from the support structure 102 exceeding 3 mm. However, in alternative embodiments it is equally conceivable that the protruding pressure element protrudes a distance P from the support structure 102 exceeding 1 mm or exceeding 5 mm or exceeding 7 mm or exceeding 10 mm.

The surface S of the protruding pressure element 101 is made from a soft inert material suitable for skin contact, which for example could be a material made from a natural fiber such as cotton, or a material made from a synthetic fiber such as a polyurethane fiber, such as Elastane.

FIG. 6 shows an alternative embodiment to the protruding pressure element shown in FIG. 5. In the protruding pressure element of FIG. 6, the surface S is part of a layer which is suspended on a first and second elastic member in the form of helical springs 105a, 105b. The helical springs 105a, 105b create an elastic pressure which enables the layer 106 to exert pressure on the surface of the skin exceeding 2 mmHg and thus compressing the veins and restricting the flow of blood therethrough. In alternative embodiments, the helical springs 105a, 105b may create an elastic pressure which enables the layer 106 to exert pressure on the surface of the skin exceeding 4 mmHg or exceeding 6 mmHg.

FIG. 7 shows an alternative embodiment to the protruding pressure element shown in FIG. 6, in which a layer 107 is a flexible layer 107 operating as an elastic member, and which is a portion of an inflatable chamber C. The inflatable chamber C is configured to be inflated by a liquid or gaseous fluid. Inflation of the inflatable chamber C creates an elastic pressure which enables the layer 107 to exert pressure on the surface of the skin exceeding 2 mmHg and thus compressing the veins and restricting the flow of blood. The inflatable chamber C is connected to a hydraulic of pneumatic fluid conduit which in turn connects the inflatable chamber C to a source of hydraulic or pneumatic pressure. In alternative embodiments, inflation of the inflatable chamber C may create an elastic pressure which enables the layer 107 to exert pressure on the surface of the skin exceeding 4 mmHg or exceeding 6 mmHg. The layer 107 is made from a soft material suitable for skin contact, such as medical grade silicone.

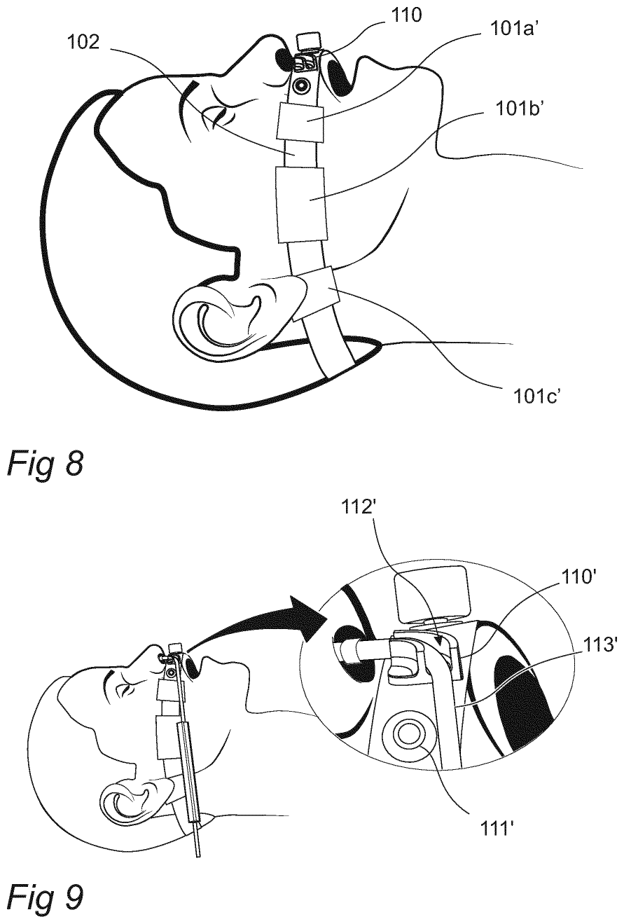

FIG. 8 shows an alternative embodiment of the constriction device 100 very similar to the embodiment shown with reference to FIG. 4. In the embodiment shown in FIG. 8, the constriction device 100 is specifically adapted to be used together with a system for hypothermia treatment of a patient by the cooling of the sinus cavernous. Such hypothermia treatment systems cool the patient by means of cooling elements adapted to be introduced through the nostrils into the nasal cavity of the patent. Such hypothermia treatment systems are further described in for example WO2005/087156 (to Lunderqvist and Allers).

In the embodiment of FIG. 8, the constriction device 100 comprises bilateral fixation elements 110′ (110″ being placed on the left side of the patient and thus not shown in FIG. 8) adapted to fixate tubing 113′ to the constriction device 100. The tubing 113′ being configured to circle a cooling fluid into a heat exchanging balloon placed in the nasal cavity of the patient. The fixation element 110′ (and the corresponding fixation element on the left side of the patient) is pivotable around a fixation point 111′, thus allowing for a flexible and comfortable fixation which is beneficial for both the patient and the medical personnel. Specifically, the one or more fixation element 110′ is removably attached to the constriction device 100 by means of a push button 111′. This provides a convenient solution where the fixation element 110′ may be varied dependent on the task.

FIG. 9 shows a more detailed view of the embodiment shown in FIG. 8. In the enlargement of the fixation element it can be seen that the fixation element 110′ comprises a recess 112′ for holding and fixating the tubing 113′. The radius of the curvature of the recess 112′ is less than 3 times a width of the recess 112′. The curved recess 112′ provides a way to fixate the tubing 113′ to the fixation element 110′ purely by frictional forces between the outer surface of the tubing 113′ and the inner surfaces of the recess 112′.

In an alternative example embodiment, a fixation element may be arranged to fixate more than one tube.

Additionally, variations to the disclosed embodiments can be understood and effected by the skilled person in practicing the claimed invention, from a study of the drawings, the disclosure, and the appended claims.

1. Method for regulating the temperature of the brain, the method comprising:
placing a constriction device on the head of a patient, the constriction device comprising at least two protruding pressure elements
wherein the constriction device further comprises a support structure wherein the protruding pressure elements are connected to the support structure,
creating bilateral pressure from the at least two protruding elements on at least two from a list consisting of:
the facial veins,
the superficial temporal veins,
the posterior auricular veins, and
the occipital veins,
and
restricting the blood flow in the constricted veins by the bilateral pressure of the at least two protruding elements.

2.-13. (canceled)

14. Method according to claim 1, wherein creating bilateral pressure comprises creating bilateral pressure on at least three from the list consisting of:
the facial veins,
the superficial temporal veins,
the posterior auricular veins, and
the occipital veins.

15. Method according to claim 1, wherein creating bilateral pressure comprises creating bilateral pressure on
the facial veins,
the superficial temporal veins,
the posterior auricular veins, and
the occipital veins.

16. Method according to claim 1, further comprising placing the support structure such that it encircles at least a portion of the head of the patient.

17. Method according to claim 1, further comprising placing the support structure such that it fully encircles the head of the patient.

18. Method according to claim 14, wherein creating bilateral pressure from the at least two protruding elements comprises creating bilateral pressure from three protruding elements.

19. Method according to claim 15, wherein creating bilateral pressure from the at least two protruding elements comprises creating bilateral pressure from four protruding elements.

20. Method according to claim 1, wherein creating bilateral pressure from the at least two protruding elements comprises creating bilateral pressure from six protruding elements.

21. Method according to claim 1, wherein creating bilateral pressure comprises creating bilateral pressure by exerting an elastic force.

22. Method according to claim 1, further comprising
adjusting the bilateral pressure by adjusting the length of the support structure.

23. Method according to claim 1, further comprising
adjusting the bilateral pressure by adjusting the at least two protruding elements in relation to each other.

24. Method according to claim 1, further comprising adjusting the bilateral pressure by adjusting the at least two protruding elements in relation to the vein which is to be compressed.

25. Method according to claim 1, wherein the temperature of the brain is regulated by creating a bilateral pressure on the veins exceeding 2 mm Hg.

26. Method according to claim 1, wherein the temperature of the brain is regulated by creating a bilateral pressure on the veins exceeding 4 mm Hg.

27. Method according to claim 1, wherein the temperature of the brain is regulated by creating a bilateral pressure on the veins exceeding 6 mm Hg.

28. Method according to claim 1, wherein the bilateral pressure is created by protruding elements protruding a distance from the support structure exceeding 3 mm.

29. Method according to claim 1, wherein the bilateral pressure is created by protruding elements protruding a distance from the support structure exceeding 5 mm.

30. Method according to claim 1, wherein the bilateral pressure is created by protruding elements protruding a distance from the support structure exceeding 10 mm.

31. Method according to claim 1, wherein the bilateral pressure is created by protruding elements being spaced apart with a distance exceeding 1 mm.

32. Method according to claim 1, wherein the bilateral pressure is created by protruding elements being spaced apart with a distance exceeding 3 mm.

33. Method according to claim 1, wherein the bilateral pressure is created by protruding elements being spaced apart with a distance exceeding 7 mm.
